(19) United States
(12) Patent Application Publication
(10) Pub. No.: US 2023/0310559 A1
(43) Pub. Date: Oct. 5, 2023

(54) Use of Amylase or Maltose to Treat or Prevent Neurodegeneration

(71) Applicant: St. Jude Children's Research Hospital, Inc., Memphis, TN (US)

(72) Inventors: Fabio DEMONTIS, Memphis, TN (US); Mamta RAI, Memphis, TN (US)

(21) Appl. No.: 18/041,873

(22) PCT Filed: Aug. 13, 2021
(86) PCT No.: PCT/US21/45843

§ 371 Date: Feb. 16, 2023

Related U.S. Application Data
(60) Provisional application No. 63/066,973, filed on Aug. 18, 2020.

(51) Int. Cl. A61K 38/47; A61K 31/7016; C12N 15/86; A61P 25/28

(52) U.S. Cl. CPC A61K 38/47; A61K 31/7016; C12N 15/86; A61P 25/28; C12Y 302/01001

(57) ABSTRACT

A method for preventing or treating a neurodegenerative disease or condition by administering to a subject in need thereof an effective amount of an amylase or maltose is provided, wherein the amylase or maltose reduces aggregation-associated or misfolded protein- associated proteotoxicity, induces transcription of chaperones and proteases, promotes degradation of proteasome substrates, or preserves protein quality under stress conditions in a subject.

This invention was made with government support under grant nos. AG055532 and AG063806 awarded by the National Institutes of Health. The government has certain rights in this invention.

### INTRODUCTION

This application claims the benefit of priority from U.S. Provisional Application Ser. No. 63/066,973, filed Aug. 18, 2020, the contents of which are incorporated herein by reference in their entireties.

### BACKGROUND

The ubiquitin-proteasome system is a fundamental pathway for normal protein turnover and for the degradation of misfolded and pathogenic proteins. Proteasome dysfunction is causally associated with many age-related pathologies including neurodegeneration. Because of its fundamental roles, there are several cellular mechanisms that monitor proteasome function and dynamically adjust its abundance, composition, and activity in response to homeostatic challenges. For example, decreased proteasome activity in yeast leads to stabilization (due to lower degradation) of the transcription factor Rpn4, which promotes the expression of proteasome components. In turn, these constitute new functional proteasomes via the action of stress-induced proteasome assembly chaperones and maturation factors. Although Rpn4 is not present in metazoans, the SKN-1/Nrf1 pathway has been shown to similarly respond to proteasome stress in multicellular organisms. However, it is largely unknown whether Nrf1-independent pathways contribute to sense and mount adaptive responses to proteasome stress.

In addition to assembling new proteasomes, an alternative mechanism to respond to local deficits in proteasome function includes the induction of other proteolytic enzymes, i.e., non-proteasomal proteases and peptidases. Specifically, cytoplasmic proteases such as tripeptidyl peptidase II (TPPII) have been shown to be induced by proteasome stress and to partially compensate for loss of proteasome function via their capacity to degrade polypeptides by exo- and endoproteolytic cleavage. In addition to normalizing protein turnover in conditions of proteasome deficits, proteases maintain protein quality control also via their capacity to degrade pathogenic proteins such as tau and huntingtin, as observed for the puromycin-sensitive aminopeptidase (PSA). However, how proteases and peptidases are modulated by proteasome stress is largely unknown.

In addition to cell-autonomous (local) responses, there is increasing evidence that stress sensing in a group of cells or a tissue induces cell-non-autonomous (systemic) adaptations. Specifically, perturbation of the unfolded protein response in a tissue is sensed systemically and induces protective responses in distant tissues. For example, expression of a misfolding-prone protein in the muscle of C. elegans induces chaperone expression in the brain and intestine via an inter-tissue signaling pathway that requires the transcription factor FOXA. Such inter-tissue stress signaling may contribute to coordinated adaptations of distinct tissues to local and systemic challenges, so that the organism can better withstand and respond to homeostatic perturbations. However, whereas proteasome stress is also sensed systemically is unknown.

The ubiquitin-proteasome system is the primary proteolytic system of skeletal muscle, which constitutes the bulk protein reserve of the organism. Skeletal muscle has emerged as an important tissue that regulates systemic aging, proteostasis, and stress responses. Such systemic effects can arise from muscle-secreted factors known as myokines, which can be modulated by stress-sensing signaling pathways. However, it is unknown whether proteasome stress in skeletal muscle is sensed systemically.

US 2006/0246155 A1 discloses a method for the treatment or prophylaxis of disorders associated with impaired mitochondrial function including disorders of the nervous system (e.g., neurodegenerative, psychoses, etc.) using a sugar; a Krebs cycle intermediate, precursor of a Krebs cycle intermediate, salt thereof, or combination thereof; and a component selected from the group consisting of an unsaturated lipid, phenylethylamine, a soluble calcium compound, or a combination thereof.

US 2019/0175703 discloses compositions composed of a glucose oxidase and at least one ingredient selected from one or more of carbohydrate (e.g., dietary fiber or saccharide), polyol or sugar alcohol for regulating one or more diseases/conditions, including but not limited to those associated with blood, kidney, thyroid, nerves, joints, weight, diabetes, oxidative stress, cardiovascular disease, insulin resistance, amyloid foot ulcers, cataract, glaucoma, hypertension, metabolic disorders, digestive disorders polycystic ovarian syndrome, mastopathy, dupuytren's contracture, gingivitis, periodontitis, dental caries, mouth disorders, cognitive dysfunction, and Parkinson's disease.

Byman, et al. ((2019) J. Alz. Dis. 68(1):205-217) teach that astrocytes respond to fibril Aβ₄₂ in Aβ plaques by increasing their α-amylase production to either liberate energy or regulate functions needed in reactive processes. It is suggested that these finding indicate that α-amylase as an important actor involved in Alzheimer's disease-associated neuroinflammation.

WO 2012/145651 A2 suggests a pharmaceutical composition for treating a disorder such as Alzheimer's disease, anxiety disorders, bipolar disorders, cognitive disorders, dementia, dissociation disorders, eating disorders, impulse regulation disorders , Mood disorders, sexual disorders, sleep disorders, psychiatric disorder which includes a therapeutically effective amount of a digestive enzyme provided as pancreatin comprising protease, amylase, and lipase.

### SUMMARY OF THE INVENTION

This invention provides a method for preventing or treating a neurodegenerative disease or condition by administering to a subject in need thereof an effective amount of an amylase or maltose. In some embodiments, the amylase is administered in the form of an isolated amylase protein. In other embodiments, the amylase is administered in the form of an amylase encoding nucleic acid molecule, i.e., inserted in a viral vector. In further embodiments, the neurodegenerative disease or condition is a neurodegenerative proteinopathy, e.g., Huntington's disease, Alzheimer's disease or Parkinson's disease. In still further embodiments, the effective amount of amylase or maltose reduces aggregation-associated or misfolded protein-associated proteotoxicity, induces transcription of chaperones and proteases, promotes degradation of proteasome substrates, or preserves protein quality under stress conditions in a subject. In yet further embodiments, the maltose is administered in the form of a pharmaceutical composition consisting of an effective amount of maltose in admixture with a suitable carrier.

### BRIEF DESCRIPTION OF THE DRAWINGS

FIG. 1 shows that pathogenic huntingtin (Htt-Q72-GFP) aggregates decrease in an age-dependent manner in response to Amyrel overexpression in Drosophila retinas compared to control mCherry and no transgene (+) overexpression. GFP-positive speckles (indicative of protein aggregates) are shown, together with the quantification of the total area and number of Htt-Q72-GFP speckles. SD is indicated with n≥118; *p<0.05 and **p<0.01.

FIG. 2 shows the quantitation of the area of poly-ubiquitin protein aggregates in the brains and retinas of flies with muscle-specific modulation of Amyrel and controls. The n and SD are indicated, with *p<0.05, **p<0.01, and ***p<0.001.

FIG. 3 shows that the capacity for startle-induced negative geotaxis (indicative of neuromuscular function) declines during aging in Drosophila. However, muscle-specific Amyrel overexpression preserves the capacity for negative geotaxis, compared to isogenic controls. Data from n[batches of 25 flies]=11 and SEM is shown.

FIG. 4 shows multielectrode array recording of neuronal activity from human cortical brain organoids treated with maltose and heat shocked (SEM with n=18 for each condition; each n represents a well with an organoid slice). Maltose treatment preserves neuronal activity, which is compromised by thermal stress. Specifically, organoids pre-treated with maltose have similar activity to control organoids in pre-heat shock conditions. However, they display a higher number of spikes, higher number of active electrodes, higher number of bursting electrodes, and more bursts compared to control-treated organoids. Overall, higher neuronal activity is found in maltose-treated organoids immediately after heat shock and also 17 and 43 hours later. The p-value 56 represents the row factor from two-way ANOVA, which indicates the effect of treatment at each time point (p<0.01). Similar results are obtained with 5 and 40 mg/mL of maltose.

### DETAILED DESCRIPTION OF THE INVENTION

It has now been found that amylase and maltose preserve protein quality control and neuronal activity in the brain, in particular in an age-dependent manner. Accordingly, this invention provides compositions and methods for preventing or treating a neurodegenerative disease or condition, in particular one in which there is accumulation of misfolded and/or aggregated proteins, by administering to a subject in need thereof an effective amount of an amylase or maltose. In some embodiments, the amylase is provided as a nucleic acid or polynucleotide encoding the amylase, as an isolated amylase protein or via a vector or host cell capable of expressing the amylase.

For the purposes of this invention, the term “neurodegenerative disease” or “neurodegenerative condition” refers to a neurodegenerative proteinopathy, which results from increased aggregation-associated or misfolded protein-associated proteotoxicity. In such diseases, aggregation or misfolding exceeds clearance inside and/or outside of the cell. Such neurodegenerative diseases are often associated with aging and include, e.g., neurodegenerative diseases associated with aggregation of polyglutamine or polyglutamine repeats, Aβ peptide, tau, transthyretin, α-synuclein, or prions. Neurodegenerative diseases associated with aggregation of polyglutamine include, but are not limited to, Huntington's disease, dentatorubral and pallidoluysian atrophy, several forms of spino-cerebellar ataxia, and spinal and bulbar muscular atrophy. Alzheimer's disease is characterized by the formation of two types of aggregates: intracellular and extracellular aggregates of Aβ peptide and intracellular aggregates of the microtubule associated protein tau. Transthyretin-associated aggregation diseases include, for example, senile systemic amyloidoses, familial amyloidotic neuropathy, and familial amyloid cardiomyopathy. Lewy body diseases are characterized by an aggregation of α-synuclein protein and include, for example, Parkinson's disease. Prion diseases (also known as transmissible spongiform encephalopathies) are characterized by aggregation of prion proteins. Exemplary human prion diseases are Creutzfeldt-Jakob Disease (CJD), Variant Creutzfeldt-Jakob Disease, Gerstmann-Straussler-Scheinker Syndrome, Fatal Familial Insomnia and Kuru. In one embodiment, said neurodegenerative disease or disorder is Huntington's disease. In another embodiment, said neurodegenerative disease or disorder is Alzheimer's disease. In a further embodiment, said neurodegenerative disease or disorder is Parkinson's disease.

The subject treated in accordance with the methods described herein can be any mammal, including, but not limited to, humans, murines, simians, felines, canines, equines, bovines, mammalian farm animals, mammalian sport animals, and mammalian pets. Ideally, the subject is a human subject.

“Treatment,” as used herein, refers to the application or administration of an agent, or pharmaceutical composition containing the agent, to a subject, isolated tissue, isolated cells or cell line from a subject, where the subject has a neurodegenerative disease or condition, or a predisposition toward development of a neurodegenerative disease or condition, where the purpose is to cure, heal, alleviate, relieve, alter, remedy, ameliorate, improve, or affect the neurodegenerative disease or condition and/or any associated symptoms of the neurodegenerative disease or condition, or the predisposition toward the development of the neurodegenerative disease or condition. Ideally, treatment of a subject, tissue or cell will reduce aggregation-associated or misfolded protein-associated proteotoxicity, induce transcription of chaperones and proteases, promote degradation of proteasome substrates, and/or preserve protein quality under stress conditions in a subject.

An “effective amount” is an amount sufficient to effect beneficial or desired results. An effective amount can be administered in one or more administrations. An effective amount corresponds with the quantity required to provide a desired average local concentration of a particular biologic agent, in accordance with its known efficacy, for the intended period of therapy. A dose may be determined by those skilled in the art by conducting preliminary animal studies and generating a dose response curve, as is known in the art. Maximum concentration in the dose response curve would be determined by the solubility of the agent in the solution and by toxicity to the animal model, as known in the art.

In some embodiments, treatment of a subject with an agent of the invention reduces levels of aggregated or misfolded proteins by at least 10% as compared to levels of aggregated or misfolded proteins in a subject that has not received treatment or in the subject within 1, 2, 3, or 4 weeks prior to the commencement of treatment of the subject. In certain embodiments, levels of aggregated or misfolded proteins are reduced in the subject by at least 25%, 30%, 33%, 35%, 40%, 45%, 50%, 55%, 60%, 65%, 70%, 75%, 80%, 85%, 90%, 95%, 98%, or 100%. A reduction in the levels of aggregated or misfolded proteins can be measured by conventional methods such as microscopic analysis, cell morphology, cognitive tests, behavioral analysis, and the like.

In other embodiments, treatment of a subject with an agent of the invention increases the transcription of chaperones and proteases/peptidases that promote the degradation of proteasome substrates. In certain embodiments, treatment of a subject with an agent of the invention increases the transcription of chaperones and proteases/peptidases by at least 10% as compared to a subject that has not received treatment or in the subject within 1, 2, 3, or 4 weeks prior to the commencement of treatment of the subject. In certain embodiments, transcription of chaperones and proteases/peptidases is increased in the subject by at least 25%, 30%, 33%, 35%, 40%, 45%, 50%, 550, 600, 65%, 704, 750, 800, 85%, 90%, 95%, 98%, or 100%. An increase in transcription of chaperones and proteases/peptidases can be measured by conventional methods such as northern blot analysis, qRT-PCR, RNA-sequencing, microarray analysis, and the like.

Agents of use in the method of this invention comprise, consist of, or consist essentially of maltose and/or amylase protein. As used herein, an amylase protein may be any of the naturally occurring isoforms or variants of an α-amylase or β-amylase. The amylase protein may be provided in accordance with the method of this invention as an isolated amylase protein or a nucleic acid molecule encoding the same. Nucleic acids containing genomic or cDNA sequences of amylase proteins are known in the art and available from GENBANK. Likewise, the amino acid sequence for numerous amylase proteins are available from GENBANK. By way of illustration, Amyrel is available under GENBANK Accession No. NP_477262.1, human AMY-1A is available under GENBANK Accession No. NP_001008222.1; human AMY-1B is available under GENBANK Accession No. NP_001008219.1; human AMY-1C is available under GENBANK Accession No. NP_001008220.1; human AMY-2A is available under GENBANK Accession No. NP_000690.10 and human AMY-2B is available under GENBANK Accession No. NP_066188.1. Amylase proteins are highly homologous with AMY-1A, AMY-1B, and AMY-1C sharing 97% sequence identity with AMY-2B and Amy-2B and AMY-2A sharing 98% sequence identity. To the extent that additional cloned sequences of amylase genes are required, they may be obtained from genomic or cDNA libraries (preferably human) using known amylase protein DNA sequences or antibodies to known amylase proteins as probes.

The amylase protein or maltose can be delivered systemically or in an organ-specific or tissue-specific manner. In some embodiments, the amylase protein or maltose is delivered to the muscles of the subject. In other embodiments, the amylase protein or maltose is delivered to the central nervous system or brain of the subject (e.g., by intracranial injection).

Muscle-specific delivery of amylase protein or maltose can be achieved by intramuscular injection, or via conjugation or fusion to a muscle-specific peptide or peptidomimetic (see, e.g., U.S. Pat. No. 8,519,097 B2). CNS or brain-specific delivery can be achieved by, e.g., conjugation to a ligand of the blood-brain barrier receptor-mediated transport (RMT) system (Pardridge (2015) Expert Opin. Ther. Targets 19:1059-107). In addition to the endogenous ligands, certain peptidomimetic monoclonal antibodies (MAb) that binds an exofacial epitope on the BBB insulin receptor (IR) or transferrin receptor (TfR) undergoes RMT across the BBB in parallel with the endogenous ligand. The MAb may act as a molecular Trojan horse to ferry into the brain any fused biologic drug that normally does not cross the BBB (Pardridge & Boado (2012) Methods Enzymol. 503:269-292). The preferred MAb Trojan horse binds a site on the BBB receptor that is spatially removed from the binding site of the endogenous ligand. The principal Trojan horse investigated in humans is a MAb against the human insulin receptor (HIR), which is designated the HIRMAb. The amylase protein can be re-engineered for BBB delivery by genetic fusion of the therapeutic protein to the heavy chain or light chain of the MAb Trojan horse (Pardridge & Boado (2012) Methods Enzymol. 503:269-292). By way of illustration, it has been shown that iduronidase fused to the carboxyl terminus of the heavy chain of the HIRMAb crosses the BBB via RMT on the endothelial IR and then undergoes receptor-mediated endocytosis into brain cells via the IR expressed on the neuronal cell membrane (Boado, et al. (2008) Biotechnol. Bioeng. 99:475-484). Similarly, glial-derived neurotrophic factor (GDNF) was fused to the carboxyl terminus of the heavy chain of the HIRMAb (Boado, et al. (2008) Biotechnol. Bioeng. 100:387-396) and shown to traverse the BBB via RMT on the IR, followed by binding of the GDNF domain of the fusion protein to the cognate neurotrophin receptor (NTR) on the neuronal cell membrane. Likewise, an Abeta amyloid single chain Fv antibody was fused to the carboxyl terminus of each heavy chain of the HIRMAb (Boado, et al. (2007) Bioconjug. Chem. 18:447-455) and shown to cause a 60% reduction in brain amyloid plaque without causing cerebral micro-hemorrhage (Sumbria, et al. (2013) Mol. Pharm. 10:3507-3513). Accordingly, in some embodiments, the amylase protein is provided in the form of a fusion protein, wherein the amylase protein is fused to the carboxyl terminus of the heavy chain of the HIRMAb.

Nucleic acids encoding amylase can be introduced into cells using any of a variety of approaches. Infection with a viral vector comprising the amylase polynucleotide is preferred. Examples of suitable viral vectors include replication defective retroviral vectors, adenoviral vectors, adeno-associated vectors and lentiviral vectors. Viral vectors for use in therapeutics typically include a virus that has been distilled or reduced to the minimum components necessary for transduction of a nucleic acid payload or cargo of interest. In this manner, viral vectors are engineered as vehicles for specific delivery while lacking the deleterious replication and/or integration features found in wild-type virus.

Ideally, expression of the amylase protein is regulated by a suitable promoter. Promoters which drive or promote expression in most tissues include, but are not limited to, human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC). Tissue-specific expression elements can be used to restrict expression to certain cell types such as, but not limited to, muscle promoters, astrocyte promoters, or nervous system promoters which can be used to restrict expression to muscle cells, neurons, astrocytes, or oligodendrocytes. Non-limiting examples of muscle-specific gene delivery include that described in US 2011/0212529, which describes muscle-specific expression vectors including muscle-specific enhancers and promoter elements derived from a muscle creatine kinase promoter and enhancers, a troponin I promoter and internal regulatory elements, a skeletal alpha-actin promoter, or a desmin promoter and enhancers. See also, e.g., Odom, et al. (2011) Mol. Ther. 19(1):36-45; Percival, et al. (2007) Traffic 8(10):1424-39; and Gregorevic, et al. (2006) Nat. Med. 12(7):787-9, which describe, in part, muscle-specific gene therapy methods. See also US 2003/0099671, which discloses a mutated rabies virus suitable for delivering a gene to a subject; U.S. Pat. No. 6,310,196, which describes a DNA construct that is useful for gene therapy; and U.S. Pat. No. 6,140,111, which disclose retroviral vectors suitable for human gene therapy in the treatment of a variety of diseases.

Non-limiting examples of nervous system-specific expression elements for neurons include neuron-specific enolase (NSE), platelet-derived growth factor (PDGF), platelet-derived growth factor B-chain (PDGF-β), synapsin (Syn), methyl-CpG binding protein 2 (MeCP2), Ca2Vcalmodulin-dependent protein kinase II (CaMKII), metabotropic glutamate receptor 2 (mGluR2), neurofilament light (NFL) or heavy (NFH), β-globin minigene preproenkephalin (PPE), enkephalin (Enk) and excitatory amino acid transporter 2 (EAAT2) promoters. Non-limiting examples of tissue-specific expression elements for astrocytes include glial fibrillary acidic protein (GFAP) and EAAT2 promoters. A non-limiting example of a tissue-specific expression element for oligodendrocytes includes the myelin basic protein (MBP) promoter. By way of illustration, low expression in all brain regions has been observed when NFL and NFH promoters are used as compared to the CMV-lacZ, CMV-luc, EF, GFAP, hENK, nAChR, PPE, PPE+wpre, NSE (0.3 kb), NSE (1.8 kb) and NSE (1.8 kb+wpre) (Xu, et al. (2001) Gene Therapy 8:1323-1332).

Retroviruses provide a convenient platform for gene delivery. A selected gene can be inserted into a vector and packaged in retroviral particles using techniques known in the art. The recombinant virus can then be isolated and delivered to cells of the subject either in vivo or ex vivo. A number of retroviral systems have been described. See, e.g., U.S. Pat. No. 5,219,740; Miller & Rosman (1989) BioTechniques 7:980-90; Miller (1990) Human Gene Therapy 1:5-14; Scarpa, et al. (1991) Virology 180:849-52; Burns, et al. (1993) Proc. Natl. Acad. Sci. USA 90:8033-37; Boris-Lawrie & Temin (1993) Curr. Opin. Genet. Develop. 3:102-09. Examples of retroviral vectors in which a heterologous gene can be inserted include, but are not limited to: Moloney murine leukemia virus (MoMuLV), Harvey murine sarcoma virus (HaMuSV), murine mammary tumor virus (MuMTV), and Rous sarcoma virus (RSV). A number of additional retroviral vectors can incorporate multiple genes. All of these vectors can transfer or incorporate a gene for a selectable marker so that transduced cells can be identified and generated. By inserting, for example, a nucleic acid encoding an amylase protein of interest into the viral vector, along with another gene which encodes a ligand for a receptor on a specific target cell, such as, for example, a muscle cell, the vector is now target specific.

Replication-defective murine retroviral vectors are widely used gene transfer vectors. Murine leukemia retroviruses include a single stranded RNA molecule complexed with a nuclear core protein and polymerase (pol) enzymes, encased by a protein core (gag), and surrounded by a glycoprotein envelope (env) that determines host range. The genomic structure of retroviruses includes gag, pol, and env genes and 5′ and 3′ long terminal repeats (LTRs). Retroviral vector systems exploit the fact that a minimal vector containing the 5′ and 3′ LTRs and the packaging signal are sufficient to allow vector packaging, infection and integration into target cells, provided that the viral structural proteins are supplied in trans in the packaging cell line. Fundamental advantages of retroviral vectors for gene transfer include efficient infection and gene expression in most cell types, precise single copy vector integration into target cell chromosomal DNA and ease of manipulation of the retroviral genome.

In some embodiments, a nucleic acid molecule encoding an amylase protein is inserted into an adenovirus-based expression vector. Unlike retroviruses, which integrate into the host genome, adenoviruses persist extrachromosomally thus minimizing the risks associated with insertional mutagenesis (Haj-Ahmad & Graham (1986) J. Virol. 57:267-74; Bett, et al. (1993) J. Virol. 67:5911-21; Mittereder, et al. (1994) Human Gene Therapy 5:717-29; Seth, et al. (1994) J. Virol. 68:933-40; Barr, et al. (1994) Gene Therapy 1:51-58; Berkner (1988) BioTechniques 6:616-29; and Rich, et al. (1993) Human Gene Therapy 4:461-76).

Adenoviral vectors for use with the present invention can be derived from any of the various adenoviral serotypes, including, without limitation, any of the over 40 serotype strains of adenovirus, such as serotypes 2, 5, 12, 40, and 41. The adenoviral vectors used herein are replication-deficient and contain the gene of interest under the control of a suitable promoter, such as any of the promoters discussed below with reference to adeno-associated virus. For example, U.S. Pat. No. 6,048,551 describes replication-deficient adenoviral vectors that can be used to express a protein of interest under the control of the Rous Sarcoma Virus (RSV) promoter. Other recombinant adenoviruses of various serotypes, and including different promoter systems, can be created by those skilled in the art. See, e.g., U.S. Pat. No. 6,306,652. Moreover, “minimal” adenovirus vectors, as described in U.S. Pat. No. 6,306,652, will find use with the present invention. Other useful adenovirus-based vectors for delivery of an amylase protein include the “gutless” (helper-dependent) adenovirus in which the vast majority of the viral genome has been removed (Wu, et al. (2001) Anesthes. 94:1119-32).

Viral vectors of the present disclosure may be further based on an adeno-associated virus (AAV) parent or reference sequence. Serotypes which may be useful in the presently disclosed compositions and methods include any of those arising from AAV1, AAV2, AAV3, AAV4, AAV5, AAV6, AAV7, AAVB, AAV9, AAV9.47, AAV9(hu14), AAV10, AAV11, AAV12, AAVrh8, AAVrh10 and AAV-DJ.

Another targeted delivery system for an agent of this invention is a colloidal dispersion system. Colloidal dispersion systems include, for example, macromolecule complexes, nanocapsules, microspheres, beads, and lipid-based systems including oil-in-water emulsions, micelles, mixed micelles, and liposomes. Liposomes are artificial membrane vesicles which are useful as delivery vehicles in vitro and in vivo. RNA, DNA and intact virions can be encapsulated within the aqueous interior and be delivered to cells in a biologically active form (see, for example, Fraley, et al. (1981) Trends Biochem. Sci. 6:77). Methods for efficient gene transfer using a liposome vehicle, are known in the art (see, for example, Mannino, et al. (1988) Biotechniques 6:682. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine and distearoylphosphatidylcholine. The targeting of liposomes is also possible based on, for example, organ-specificity, cell-specificity, and organelle-specificity and is known in the art.

In some embodiments, cells expressing an amylase protein can be delivered by direct application, for example, direct injection of a sample of such cells into a target site, such as muscle tissue thereby delivering the amylase protein. These cells can be purified. In some embodiments, such cells can be delivered in a medium or matrix which partially impedes their mobility so as to localize the cells to a target site. Such a medium or matrix could be semi-solid, such as a paste or gel, including a gel-like polymer. Alternatively, in some embodiments, the medium or matrix could be in the form of a solid, a porous solid which will allow the migration of cells into the solid matrix, and hold them there while allowing proliferation of the cells.

In other embodiments, an amylase protein is delivered to a cell or administered to a subject in the form of a modified RNA encoding the amylase protein. A modified RNA encoding the amylase protein described herein can include a modification to prevent rapid degradation by endo- and exo-nucleases and/or to avoid or reduce the cell's innate immune or interferon response to the RNA. Modifications include, but are not limited to, e.g., (a) end modifications such as 5′-end modifications (phosphorylation dephosphorylation, conjugation, inverted linkages, etc.) and 3′-end modifications (conjugation, DNA nucleotides, inverted linkages, etc.); (b) base modifications, e.g., replacement with modified bases, stabilizing bases, destabilizing bases, or bases that base pair with an expanded repertoire of partners, or conjugated bases; (c) sugar modifications (e.g., at the 2′ position or 4′ position) or replacement of the sugar; and/or (d) internucleoside linkage modifications, including modification or replacement of the phosphodiester linkages. To the extent that such modifications interfere with translation (i.e., results in a reduction of 50% or more in translation relative to the lack of the modification (e.g., in a rabbit reticulocyte in vitro translation assay), the modification is not suitable for the methods and compositions described herein. Specific examples of modified RNA compositions useful with the methods described herein include, but are not limited to, RNA molecules containing modified or non-natural internucleoside linkages. Modified RNAs having modified internucleoside linkages include, among others, those that do not have a phosphorus atom in the internucleoside linkage. In other embodiments, the modified RNA has a phosphorus atom in its internucleoside linkage(s). Non-limiting examples of modified internucleoside linkages include phosphorothioates, chiral phosphorodithioates, phosphorothioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Modified internucleoside linkages that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Another modification for use with the synthetic, modified RNA described herein involves chemically linking to the RNA one or more ligands, moieties or conjugates that enhance the activity, cellular distribution or cellular uptake of the RNA. Ligands can be particularly useful where, for example, a synthetic, modified RNA is administered in vivo. Such moieties include but are not limited to lipid moieties such as a cholesterol moiety (Letsinger, et al. (1989) Proc. Natl. Acid. Sci. USA 86:6553-6556), cholic acid (Manoharan, et al. (1994) Biorg. Med. Chem. Let. 4:1053-1060), a thioether, e.g., beryl-S-tritylthiol (Manoharan, et al. (1992) Ann. NY Acad. Sci. 660:306-309; Manoharan, et al. (1993) Biorg. Med. Chem. Lett. 3:2765-2770), a thiocholesterol (Oberhauser, et al. (1992) Nucl. Acids Res. 20:533-538), an aliphatic chain, e.g., dodecandiol or undecyl residues (Saison-Behmoaras, et al. (1991) EMBO J. 10:1111-1118; Kabanov, et al. (1990) FEBS Lett. 259:327-330; Svinarchuk, et al. (1993) Biochimie 75:49-54), a phospholipid, e.g., di-hexadecyl-rac-glycerol or triethyl-ammonium 1,2-di-O-hexadecyl-rac-glycero-3-phosphonate (Manoharan, et al. (1995) Tetrahedron Lett. 36:3651-3654; Shea, et al. (1990) Nucl. Acids Res. 18:3777-3783), a polyamine or a polyethylene glycol chain (Manoharan, et al. (1995) Nucleosides & Nucleotides 14:969-973), adamantane acetic acid (Manoharan, et al. (1995) Tetrahedron Lett. 36:3651-3654), a palmityl moiety (Mishra, et al. (1995) Biochim. Biophys. Acta 1264:229-237), or an octadecylamine or hexylamino-carbonyloxycholesterol moiety (Crooke, et al. (1996) J. Pharmacol. Exp. Ther. 277:923-937).

The modified RNA encoding a amylase protein described herein can further include (i) a 5′ cap, e.g., 5′ diguanosine cap, tetraphosphate cap analogs having a methylene-bis(phosphonate) moiety, dinucleotide cap analogs having a phosphorothioate modification, cap analogs having a sulfur substitution for a non-bridging oxygen, N7-benzylated dinucleoside tetraphosphate analogs, or anti-reverse cap analogs; (ii) a 5′ and/or 3′ untranslated region (UTR), e.g., a UTR from an mRNA known to have high stability in the cell (e.g., a murine alpha-globin 3′ UTR); (iii) a Kozak sequence; and/or (iv) a poly (A) tail of, e.g., at least 5 adenine nucleotides in length and can be up to several hundred adenine nucleotides.

A nucleic acid molecule encoding an amylase protein (e.g., DNA vector or RNA) can be introduced into a cell in any manner that achieves intracellular delivery of the nucleic acid molecule, such that expression of the polypeptide encoded by the nucleic acid molecule can occur. As used herein, the term “transfecting a cell” refers to the process of introducing nucleic acids into cells using means for facilitating or effecting uptake or absorption into the cell, as is understood by those skilled in the art. Absorption or uptake of a nucleic acid molecule can occur through unaided diffusive or active cellular processes, or by auxiliary agents or devices. Exemplary methods for introducing a nucleic acid molecule into a cell include, for example, transfection, nucleofection, lipofection, electroporation (see, e.g., Wong & Neumann, (1982) Biochem. Biophys. Res. Commun. 107:584-87), microinjection (e.g., by direct injection of the nucleic acid molecule), biolistics, cell fusion, and the like. In an alternative embodiment, a nucleic acid molecule can be delivered using a drug delivery system such as a nanoparticle, a dendrimer, a polymer, a liposome, or a cationic delivery system. Positively charged cationic delivery systems facilitate binding of a nucleic acid molecule (negatively charged polynucleotides) and also enhances interactions at the negatively charged cell membrane to permit efficient cellular uptake. Cationic lipids, dendrimers, or polymers can either be bound to the nucleic acid molecule, or induced to form a vesicle or micelle (see e.g., Kim, et al. (2008) J. Contr. Rel. 129(2):107-116) that encases the nucleic acid molecule.

In embodiments involving in vivo administration of a nucleic acid molecule encoding an amylase protein or compositions thereof, the nucleic acid molecule is formulated in conjunction with one or more penetration enhancers, surfactants and/or chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycolic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether.

Ideally, the agents of the present invention (i.e., maltose, or amylase proteins or nucleic acids encoding the same) are formulated together with a pharmaceutically acceptable carrier and provided as a pharmaceutical composition. Pharmaceutical formulations comprising the agents of the invention may be prepared for storage by mixing the agent having the desired degree of purity with optional physiologically acceptable carriers, excipients or stabilizers (Remington: The Science and Practice of Pharmacy 20^{th} edition (2000)), in the form of aqueous solutions, lyophilized or other dried formulations. Therefore, the invention further relates to a lyophilized or liquid formulation containing amylase (amylase protein or nucleic acids encoding the same) or maltose. As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for the desired route of administration. These include, but are not limited to, enteral (into the intestine), gastroenteral, epidural (into the dura matter), intracerebral (into the cerebrum), intracerebroventricular (into the cerebral ventricles, a.k.a. “intraventricular”), intradural (within or beneath the dura), intrastriatal (within the striatum, caudate nucleus and/or putamen), peridural, epicutaneous (application onto the skin), subcutaneous (under the skin), intradermal (into the skin itself), transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, nasal administration (through the nose), intravenous (into a vein), intravenous bolus, intravenous drip, intraarterial (into an artery), intramuscular (into a muscle), intracardiac (into the heart), intraosseous infusion (into the bone marrow), intraperitoneal, (infusion or injection into the peritoneum), intravesical infusion, intravitreal, (through the eye), parenteral, percutaneous, periarticular, perineural, periodontal, submucosal, topical, or spinal. In some embodiments, compositions may be administered in a way which allows them cross the blood-brain barrier (BBB), vascular barrier, or other epithelial barrier. Depending on the route of administration, the agent may be coated in a material to protect the agent from the action of acids and other natural conditions that may inactivate the agent.

A pharmaceutical composition of the invention also may include a pharmaceutically acceptable antioxidant. Examples of pharmaceutically acceptable antioxidants include water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydro xyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of an injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active agent in the required amount in an appropriate solvent with one or a combination of ingredients enumerated above, as required, followed by sterilization micro filtration. Generally, dispersions are prepared by incorporating the active agent into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active agent plus any additional desired ingredient from a previously sterile-filtered solution thereof.

The amount of agent that can be combined with a carrier material to produce a single dosage form will vary depending upon agent, the subject being treated, and the particular mode of administration. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. In accordance with this invention, daily doses of may be in the range of about 0.01 mg to 100 mg, or about 0.1 to 50 mg. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active agent and the particular therapeutic effect to be achieved, and the limitations inherent in the art of compounding such an active compound for the treatment of sensitivity in individuals.

In certain embodiments, AAV particle or viral vector pharmaceutical compositions may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic or prophylactic effect. It will be understood that the above dosing concentrations may be converted to vg or viral genomes per kg or into total viral genomes administered by one of skill in the art.

Alternatively, the agents of the invention can be administered as a sustained release formulation in which case less frequent administration is required. Dosage and frequency vary depending on the half-life of the compounds in the patient.

Actual dosage levels of the active compounds in the pharmaceutical compositions of the present invention may be varied so as to obtain an amount of the active compounds which is effective to achieve the desired therapeutic response for a particular patient, composition, and mode of administration, without being toxic to the patient. The selected dosage level will depend upon a variety of pharmacokinetic factors including the activity of the particular agent of the present invention employed, the route of administration, the time of administration, the rate of excretion of the particular agent being employed, the duration of the treatment, other drugs, compounds and/or materials used in combination with the particular compositions employed, the age, sex, weight, condition, general health and prior medical history of the patient being treated, and like factors well known in the medical arts.

The desired dosage may be delivered three times in a single day, two times in a single day, once in a since day or in a period of 24 hours the dosage may be delivered once, twice, three times or more than three times. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens may be used. As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24-hour period. It may be administered as a single unit dose. In one embodiment, the AAV particles or viral vectors of the present disclosure are administered to a subject in split doses, and may be formulated in buffer only or in a formulation described herein.

In certain embodiments, a pharmaceutical composition of use in this invention consists of or consists essentially of an effective amount of maltose as the active agent in admixture with a suitable carrier. In some embodiments, amounts of maltose that may be administered achieve a level of maltose in the brain of 0.1 mg/mL to 20 mg/mL, or preferably 1 mg/mL to 10 mg/mL or more preferably about 5 mg/mL.

In addition to treatment, this invention finds use in improving the stress resistance and cellular activity of cultured organoids (e.g., the brain organoids described herein), which are experimental models for testing drugs or conditions that are otherwise stressful.

The following non-limiting examples are provided to further illustrate the present invention.

### Example 1: Materials and Methods

Whole-Mount Immunostaining of Drosophila Brains and Retinas. The immunostaining of brains and retinas was carried out using established methods (Demontis & Perrimon (2009) Development 136(6):983-93; Demontis & Perrimon (2010) Cell 143(5):813-825). In brief, retinas and brains were dissected, fixed for 30 minutes in phosphate-buffered saline (PBS) with 4% paraformaldehyde and 0.2% TRITON⋅ X-100 at room temperature, washed for >3 times in PBS with 0.2% TRITON⋅ X-100 at room temperature, and immunostained overnight at 4° C. with rabbit anti-poly-ubiquitin (FK2; Enzo Life Sciences) and anti-Ref(2)P/p62 antibodies (Abeam). After washes with PBS with 0.2% TRITON⋅ X-100, the samples were incubated for 2 hours at room temperature with secondary antibodies and phalloidin conjugated to a fluorescent dye sold under the tradename ALEXA FLUOR® 635. The samples were subsequently washed, mounted in antifade medium and imaged on a Zeiss LSM 880 confocal microscope.

Whole-Mount Immunostaining of Human Cortical Brain Organoids. Similar to the above-referenced procedures described for Drosophila tissues, human cortical brain organoids were immunostained with the following modifications: fixation and washes were done with 0.4% TRITON⋅ X-100; fixation was carried out for 50 minutes at room temperature; and organoids were blocked for >1 hour with PBS with 0.4% TRITON X-100 and 5% bovine serum albumin (BSA) before incubation with primary antibodies.

Cloning of Myokines and Fly Transgenesis. The coding sequences of Drosophila CG4666, CG8093, Amyrel, Ag5r2, Amy-d, and Amy-p were generated as synthetic minigenes and cloned into the pUASTattB vector with the EcoRI and KpnI restriction enzymes. These plasmids were then injected into attP40 and/or attP2 by using the site-specific phiC31 integrase (Markstein, et al. (2008) Nat. Genet. 40(4):476-483) to generate transgenic flies.

Amylase Activity Assay. Colorimetric detection of amylase activity was performed according to the manufacturer's instructions (Sigma-Aldrich). In brief, five flies were decapitated and placed on ice in a 1.5 ml tube. The decapitated bodies were homogenized in 150 μL of Amylase Assay Buffer (AAB). The homogenate was collected after high-speed centrifugation for 10 minutes. For this assay, 5 μL the body homogenate or standard was added to a well in a 96-well plate and brought up to 50 μL with AAB and water, respectively. A master reaction mix was made to conduct a colorimetric assay, and 100 μL was added to each well. The plate was mixed well using a horizontal shaker. After 2-3 minutes the absorbance was measured at 405 nm. The plate was incubated at 25° C., measuring the absorbance at 405 nm every 5 minutes until the value of the most active sample exceeded the value of the highest standard.

Maltose Assays. Flies were decapitated and placed in a perforated 0.6 mL tube resting in a 1.5 mL tube, which was centrifuged at 1700 g for 6 minutes at 4° C. to collect the hemolymph (Geminard, et al. (2009) Cell Metab. 10:199-207). Equal volumes of hemolymph (1 μL) were used for subsequent determination of maltose levels.

To avoid interference from eye pigments (Al-Anzi, et al. (2010) PLoS ONE 5(8):e12353), flies (n=10/biological replicate) were decapitated and homogenized with a Next Advance BULLET BLENDER® and 0.5-mm zirconium beads in 100 μL of maltose assay buffer (MAB). Body homogenates were then collected after centrifugation at maximum speed for 10 minutes to remove cuticle debris. Subsequently, the quantification of maltose concentration in the hemolymph and bodies was done with the maltose assay kit (Sigma-Aldrich), according to manufacturer's instructions. In brief, hemolymph and body homogenates were diluted 1:40 in MAB. A 2.5 μL portion of the sample or of the standard were added per well in a 96-well plate. Then, 1 μL of α-D-glucosidase was added to standards and samples to estimate maltose levels based on degradation to glucose. In parallel, 1 μL of MAB was added to duplicate samples to estimate glucose background levels in the absence of glucosidase treatment. Subsequently, 46.5 μL of reaction mix (44.5 μL MAB, 1 μL maltose probe, 1 μL maltose enzyme mix) were added to each well. The plate was then incubated at 37° C. shielded from light. After 1 hour, the absorbance was read at 570 nm with a Tecan Infinite 200 Pro. Blank (0 maltose standards) readings were subtracted from all readings. Maltose levels were calculated from the standard curve after subtracting the absorbance read in the absence of glucosidase treatment, which accounts for background glucose levels, from the absorbance read in duplicate samples treated with glucosidase.

Western Blots of Detergent-Soluble and Detergent-Insoluble Fractions. Western blots for detergent-soluble and detergent-insoluble fractions were carried out as previously described (Demontis & Perrimon (2010) Cell 143(5):813-825). In brief, heads and thoraces were dissected from 30 male flies/sample and homogenized in ice-cold PBS with 1% TRITON™ X-100 containing protease and phosphatase inhibitors. Homogenates were centrifuged at 14,000 rpm at 4° C. and supernatants collected (TRITON™ X-100-soluble fraction). The remaining pellet was washed in ice-cold PBS with 1% TRITON™ X-100. The pellet was then resuspended in RIPA buffer containing 8M urea and 5% SDS, centrifuged at 14,000 rpm at 4° C., and the supernatant collected (TRITON™ X-100-insoluble fraction). Detergent-soluble and detergent-insoluble fractions were analyzed on 4-20% SDS-PAGE with anti-ubiquitin (Cell Signaling Technologies P4D1), anti-Ref(2)P/p62 (Abcam), anti-Atg8/GABARAP (Abcam), and/or anti-GFP (Cell Signaling Technologies) antibodies. Ponceau S staining, anti-Tubulin antibodies (Cell Signaling Technologies), and/or anti-actin antibodies were used as loading controls.

Analysis of Pathogenic Huntingtin Aggregation. Pathogenic Huntingtin-polyQ72-GFP protein aggregates (Zhang, et al. (2010) Genetics 184(4):1165-79) were imaged with an epifluorescence microscope and the number and total area of protein aggregates estimated with CELLPROFILER™. This analysis was done with male flies after aging at 25° C. for 30 days, and/or aging at 29° C. for 20 days. A custom-made script was used for image analysis in CELLPROFILER™.

Soluble and Insoluble Protein Fractionation from Drosophila Tissues. Thoraces or heads were dissected from 30 male flies and homogenized in 100 μL ice-cold TRITON™ X-100 buffer (1% TRITON™ X-100 in PBS containing protease inhibitor and phosphatase inhibitor) for 5 minutes at highest speed. Homogenates were centrifuged at 14000 rpm at 4° C. for 10 minutes and supernatant collected (TRITON™ X-100-soluble fraction). The remaining pellet was washed in 400 μL of TRITON™ X-100 buffer, twice at 14000 rpm for 5 minutes at 4° C. The pellet was then resuspended in 100 μL 1× RIPA buffer containing 8M urea and 5% SDS at room temperature, centrifuged at 14000 rpm at 4° C. for 10 minutes, and supernatant was collected (TRITON™ X-100-insoluble fraction). Six μL of soluble/insoluble protein extracts were boiled with sample buffer containing DTT and separated by gel electrophoresis.

Soluble and Insoluble Protein Fractionation from Cortical Organoids. Protein extraction from cortical organoids was performed as described for Drosophila tissues with following changes. Cortical organoids were washed with PBS and centrifuged at 2000 g for 5 minutes to remove growth medium. Buffer (50 μL) was added to the pellet and the pellet was homogenized for 30 seconds. Five μg of protein was boiled with sample buffer containing DTT and separated by gel electrophoresis.

Soluble and Insoluble Protein Fractionation from HET-293 Cells. Using a modified protocol (Holden & Horton (2009) BMC Res. Notes 2:243), soluble and insoluble protein fractions were obtained from HEK-293 cells. The HEK293 cell pellet was resuspended in ice-cold TRITON™ X-100 buffer (1% TRITON™ X-100 in PBS containing protease inhibitor and phosphatase inhibitor) by pipetting up and down. The cell suspension was centrifuged at 14000 rpm to pellet the cells. The supernatant was carefully pipetted without disturbing the pellet (TRITON™ X-100-soluble fraction). The cell pellet was washed twice in ice cold TRITON™ X-100 buffer and resuspended in urea buffer (7M Urea+1×RIPA+100U/mL Benzonase) and allowed to incubate at room temperature for 15-20 minutes to allow digestion of genomic DNA. Subsequently, a SDS solution was added to a final concentration of 1%. The samples were homogenized (without beads) at maximum speed for 30 seconds. The samples were then centrifuged at 14000 rpm for 10 minutes to pellet any remaining cell debris. The resulting supernatant was the TRITON™ X-100-insoluble extract. Five μg of protein was boiled with sample buffer and DTT and separated by gel electrophoresis.

siRNA Transfection of HEK293 Cells. Approximately 200,000 HEK293 cells were seeded in a 6-well plate. Twenty-four hours later, the cells were transfected with siRNA using a transfection reagent sold under the tradename LIPOFECTAMINE® 2000 to a final concentration of 200 nM siRNA per well. For double siRNA transfection, 150 nM siRNA each was used, totaling 300 nM siRNA per well. After 24 hours, transfected cells were fed with medium supplemented with 10% FBS and 50 mg maltose per well in a total volume of 5 mL. After 40 hours of maltose supplementation, duplicate plate was heat-shocked for 7 hours at 41.5° C.

Analysis of Huntingtin-GFP Aggregates in Drosophila Eye. Frozen Drosophila was used for imaging of the eyes using ZEISS SteREO Discovery.V12 microscope at a specific exposure time and consistent settings. The acquired gray scale images were then analyzed for the area occupied by aggregates (Huntingtin-GFP specks) using CELLPROFILER™ 3.0.0 pipeline.

Startle-Induced Negative Geotaxis. Startle-induced negative geotaxis was carried out according to established methods. Specifically, flies were tapped and the number of flies that reached the top of the vial was scored after 20 seconds.

qRT-PCR. qRT-PCR was performed as previously described (Demontis, et al. (2014) Cell Reports 7(5):1481-94). Total RNA was extracted with the TRIZOL™ reagent (Life Technologies) from Drosophila heads and thoraces from >30 male flies/replicate, followed by reverse transcription with the iSCRIPT™ cDNA synthesis kit (Bio-Rad). qRT-PCR was performed with a fluorescent dye sold under the tradename SYBR® Green and a CFX96 apparatus (Bio-Rad). Three biological replicates were used for each genotype and time point. α-Tubulin at 84B was used as a normalization reference. The comparative CT method was used for relative quantitation of mRNA levels.

RNA-Sequencing. Total RNA was extracted from Drosophila thoraces, which are composed primarily of skeletal muscle. Three or more biological replicates were prepared for RNA-seq with the TRUSEQ® stranded mRNA library preparation kit (Illumina) and sequenced on the Illumina HiSeq 4000 platform, with six samples in each lane. Multiplexing was done on a per flowcell basis. Approximately 100 million reads were obtained for each sample. FASTQ sequences derived from mRNA paired-end 100-bp sequences were mapped to the Drosophila melanogaster genome (BDGP5 release 75) with the STAR aligner (version 2.5.3a; Dobin, et al. (2013) Bioinformatics 29(1):15-21).

Transcripts were counted using HTSeq (version 0.6.1p1; Anders, et al. (2015) Bioinformatics 31(2):166-9) based on the BDGP5 GTF release 75. Log₂ (FPKM) values were calculated, imported into Partek Genomic Suite 6.6, and visualized by PCA.

False Discovery Rate (FDR) and p values for RNA-seq data were determined using Partek. Read counts for RNA-seq data were imported into R 3.2.3, TMM normalized (Robinson & Oshlack (2010) Genome Biol. 11:R25), and fitted by voom to linear models (Law, et al. (2014) Genome Biol. 15:R29). Statistical tests and FOR were derived from contrasts and refined with the eBayes function as implemented in the lirnma package in R 3.2.3 (Ritchie, et al. (2015) Nucleic Acids Res. 43(7):e47). The gene sets were analyzed by DAVID (Database for Annotation Visualization and Integrated Discovery) to identify enriched functional classes of genes.

RNA-Sequencing of Human Cortical Brain Organoids. RNA sequencing libraries for each sample were prepared with 1 μg total RNA using the Illumina TRUSEQ® RNA Sample Prep v2 Kit per the manufacturer's instructions, and sequencing was completed on the Illumina HiSeq 2000. The 100 bp paired-end reads were trimmed, filtered against quality (Phred-like Q20 or greater) and length (50 bp or longer), and aligned to a human reference sequence GRCh38 (UCSC hg38), using CLC Genomics Workbench v12.0.1 (Qiagen). For gene expression comparisons, the TPM (transcript per million) counts were obtained from the RNA-Seq Analysis tool. A total of 19749 genes with TPM greater than one in at least one sample were in the principal component analysis (PCA) and the gene expression analysis was performed using the non-parametric ANOVA using the Kruskal-Wallis and Dunn's tests on log-transformed TPM counts between three replicates of each experimental group, implemented in Partek Genomics Suite v7.0 software (Partek Inc.). The expression of a gene was considered significantly different if the adjusted P-value was less than 0.05 and the expression change was more than two-fold in at least one of the group comparisons. The z-scores of 2095 significantly differential expressed genes were calculated and hierarchical clustered in a heat map, using correlation distance measure, implemented in Spotfire v7.5.0 software (TIBCO).

Other Computational Analyses. Amyrel promoter analysis was initially done with Genomatix, using the 1 kb proximal region (chr2R:16652699. 16653699 from 31 dm6). Subsequent analyses further defined the presence of C/EBP binding sites in the Amyrel promoter region by using FIMO (--thresh 1e-3 --motif-pseudo 0.0001 --no-qvalue) from MEME suite (version 4.11.3) to scan the collection of C/EBP-related motifs (from TRANSFAC) on the Amyrel gene promoter.

Drosophila S2R+Cell Culture. One million S2R+ cells were seeded in 3 mL of Schneider's cell culture medium (Gibco) containing heat-inactivated 10% fetal calf serum (FCS) in each well of a six-well plate, with the addition of either 100 mg/mL of maltose, 100 mg/mL of glucose, 1.7 mg/mL of porcine amylase, or mock treatments. After culturing at 25° C. for 3 days, the cells were kept for 6 hours at 37° C. for heat shock. Subsequently, the cells and cell culture medium were scraped and centrifuged at maximum speed for 5 minutes, the supernatant was removed, and the cell pellet was frozen. The cell pellet was then processed for the western blot analysis of detergent-soluble and detergent-insoluble fractions.

HEK293T Cell Culture. HEK293T cells were obtained from the ATCC and cultured according to established methods. Specifically, cells were maintained at 37° C. with 5% CO₂ in DMEM containing 10% fetal bovine serum, GLUTAMAX™ and penicillin/streptomycin. HEK293T cells were screened regularly to ensure the absence of mycoplasma. For experimental treatment, 200,000 HEK293 cells were plated in 2 mL in each well of a 6-well plate. After ˜30 minutes, an additional 3 mL of culture medium was added to reach the final amount of 0, 15, 25, 50, and 100 mg of maltose per well (5 mL). After culturing at 37° C. for 3 days, the cells were kept for 7 hours at 41.5° C. for heat shock, or at 37° C. (control). Subsequently, the cells were scraped and centrifuged at maximum speed for 5 minutes, the supernatant removed, and the cell pellet frozen. The cell pellet was then processed as described above for the western blot analysis of detergent-soluble and detergent-insoluble fractions.

Human Embryonic Stem Cell (ESC) Culture. H9/WA09 cells (WiCell) were cultured on hES-qualified cell culture matrix sold under the tradename MATRIGEL® (Corning) in complete mTeSR™1 (STEMCELL Technologies) at 5% O₂, 37° C. and 5% CO₂. The cultures were passaged with EDTA (sold under the tradename VERSENE®, ThermoFisher).

Generation of Human Cortical Brain Organoids. Human cortical brain organoids were generated using established methods (Kadoshima, et al. (2013) Proc. Natl. Acad. Sci. USA 110:20284-20289; Lancaster, et al. (2013) Nature 501:373-379). Briefly, H9 cultures were dissociated into single cells with a cell detachment enzyme sold under the trademark ACCUTASE® (Innovative Cell technologies), and plated into low-attachment 96-well V-bottom plates (Sbio) at 9000 cells/well, in EB media (DMEM: F12, 20% Knockout Serum Replacement (Life Technologies), 3% ES-FBS (SIGMA), 100X GLUTAMAX™ (Gibco), 1000× β-mercaptoethanol (Gibco), 100× antibiotic-antimycotic (Gibco)) supplemented with 5 μM SB-431542 (4-[4-(1,3-benzodioxok-5-yl)-5-(2-pyridinyl)-1H-imidazol-2-yl]benzamide, TGF-β inhibitor, Tocris), 2 μM Dorsomorphin (Tocris), 3 μM IWR1e (Wnt inhibitor, EMD Millipore), and 20 μM Y-27632 (ROCK inhibitor, Stemcell technologies). Half media was replaced on day 2. On days 4, 6, and 8, half media was replaced with Glasgow Minimum Essential Medium (GMEM) KNOCKOUT™ Serum Replacement (KSR) media (GMEM, 20% KSR, 100× non-essential amino acids (Gibco), 100× Pyruvate (Gibco), 500× β-mercaptoethanol, 100X antibiotic-antimycotic) supplemented with 5 μM SB-431542, 3 μM IWR1e, 2.5 μM Cyclopamine (Stemcell technologies) and 20 μM ROCKi. On days 10, 12, and 14, half media was replaced with GMEM KSR media supplemented with 5 μM SB-431542, 3 μM IWR1e, and 20 μM Y-27632. The same medium replacement was carried out on day 16, but without Y-27632. On days 18 and 20, half media was replaced with CBO N2 media (DMEM:F12, 100× chemically defined lipid concentrate (Life technologies), 100× N2 supplement (Gibco) and 100X antibiotic-antimycotic). On day 22, organoids were transferred to a mini-bioreactor (ABLE Corporation, Tokyo) in CBO N2 media supplemented with 50× B27 supplement without Vitamin A (Gibco), 20 ng/mL bFGF (Stemcell technologies) and 20 ng/mL Epidermal Growth Factor (EGF, Peprotech), and centrifuged at 55 rpm. Media was replaced on days 24, 26, and 28. On day 30, media was changed to CBO PBS media (DMEM:F12, 100× chemically defined lipid concentrate (Life technologies), 100× N2 supplement (17502-048, Gibco), 10% ES-FBS, 5 μg/mL Heparin and 100× antibiotic-antimycotic) supplemented with 1% v/v growth factor reduced-cell culture matrix sold under the tradename MATRIGEL® (Corning), 100× N2 supplement and 50× B27 supplement without Vitamin A, and replaced every 3 days until day 39. From day 42 to 48, media was changed to CBO FBS media supplemented with 50× B27 supplement without Vitamin A, 20 ng/mL brain derived neurotrophic factor (BDNF, Peprotech) and 10 ng/mL Glial-Derived Neurotrophic Factor (GDNF, Peprotech), and replaced every 3 days. Day 51 onwards, media was changed to BrPhys media (Stemcell technologies) supplemented with 100× N2 supplement, 50X B27 supplement without Vitamin A, 10 ng/mL BDNF and 10 ng/mL GDNF, and replaced every 3-4 days. After day 70, large cortical organoids were pinched off into 2-3 smaller pieces using a pair of sterile forceps, in order to avoid large necrotic centers.

Drug Treatment of Human Cortical Brain Organoids. An additional 0.5 mL of culture medium was added to reach the final amount of 1 mL with 0, 3, 5, 10, 20, 40, or 80 mg of maltose per mL, or with 10, 20, 40 μL of porcine alpha-Amylase (MP Biomedicals) per mL, as indicated. After culturing at 37° C. for 3 days, the organoids were kept for 7 hours at 41.5° C. for heat shock, or at 37° C. (control). Subsequently, the organoids were collected and snap frozen. The organoids were then processed as described above for the western blot analysis of detergent-soluble and detergent-insoluble fractions.

Whole-mount immunostaining of organoids was carried out following similar procedures as described above for Drosophila brains and retinas, with anti-poly-ubiquitin (FK2; Enzo Life Sciences) and anti-p62/SQSTM1 antibodies (Cell Signaling Technologies). F-actin and nuclei were stained with phalloidin conjugated to a fluorescent dye sold under the tradename ALEXA FLUOR® 635 (1:100; Invitrogen) and DAPI (1 μg/mL), respectively. Organoids were mounted on slides with spacers and imaged with a Zeiss LSM 880 confocal microscope. In all cases, the peripheral regions of organoids were imaged.

Multielectrode Array Recording of Human Cortical Brain Organoids. Spontaneous neuronal activity of cortical organoids was recorded on the Axion Maestro Edge multielectrode array system. Two different methods for plating and recording were optimized from two-month old and three-month old organoids, to ensure maximum contact with electrodes.

Method 1 (Two-month old cortical organoids; Maltose treatment): Growth factor reduced-cell culture matrix sold under the tradename MATRIGEL® (Corning) was diluted 1:3 in chilled media containing BrainPhys and 10% ES-FBS and kept on ice. D60 cortical organoids were first sliced in half using a pair of fine tweezers. Each half was then plated onto one well of a 24-well Cytoview plate (Axion Biosystems), where each well contained a grid of 16 electrodes. After placing the organoid slice onto a dry and empty well using a sterile flat spatula, 15 μL of the chilled and diluted cell culture matrix sold under the tradename MATRIGEL® was added onto the slice. A P10 tip was used to position the slice to the center of the well over the grid of electrodes. Three such plates were prepared. The plates were incubated at 37° C. for 3 hours for the cell culture matrix to solidify over the organoid slices. After 3 hours, 500 μL of BrainPhys media supplemented with 100× N2 supplement, 50× B27 supplement without Vitamin A, 10 ng/ml BDNF and 10 ng/ml GDNF were added slowly to each well. Every three days, 300 μL media in each well was replaced with fresh media three days and six days after plating. Seven days after plating, the plates were treated with either control or maltose containing media; all treatment conditions were included in each of the four plates. Three days after treatment, spontaneous activity was recorded from each plate. The plates were then subjected to heat shock for 7 hours at 41.5° C. Spontaneous activity in each plate was recorded again after the heat shock. This recording was repeated after 17 hours of recovery and after 43 hours of recovery. Data was analyzed using the Neural Metric tool (Axion Biosystems).

Example 2: Muscle-Specific RNAi for Proteasome Components Systemically Improves the Degradation of Proteasome Substrates in Distant Tissues

In Drosophila, a common strategy to induce proteasome stress includes in the use of transgenic RNAi to target components of the proteasome (Tsakiri, et al. (2019) Autophagy 15(10):1757-73; Lundgren, et al. (2005) Mol. Cell Biol. 25(11):4662-75; Wojcik, et al. (2002) J. Cell Sci. 117:281-292). To investigate whether local perturbation of the proteasome in skeletal muscle induces a compensatory stress response, RNAi for Prosβ1 (a component of the 20S proteasome catalytic core) was driven in thoracic muscles via the UAS/Gal4 system and the skeletal muscle-specific Mhc-Gal4 driver (Schuster, et al. (1996) Neuron 17(4):641-654; Demontis & Perrimon (2010) Cell 143(5):813-825), and RNAi was confirmed by qRT-PCR. RNA-seq analyses revealed the induction of compensatory transcriptional changes in muscle in response to Prosβ1^{RNAi}, compared to control white^{RNAi}. Specifically, Prosβ1^{RNAi} increased the expression of chaperones and proteases/peptidases, which promote the degradation of proteasome substrates and are induced by proteasome stress in other systems (Geier, et al. (1999) Science 283(5404):978-81; Glas, et al. (1998) Nature 392:618-22). Similar transcriptional stress responses were also found with muscle-specific Prosβ5^{RNAi} and RNAi for Ter94, the Drosophila homolog of p97/VCP (valosin-containing protein) which cooperates with the proteasome in the degradation of proteins from organellar membranes and multimolecular complexes (Piccirillo & Goldberg (2012) EMBO J. 31(15):3334-50; van den Boom, et al. (2016) Mol. Cell 64(1):189-198). Together, these findings indicate the induction of a local, transcriptional response to proteasome stress in skeletal muscle.

To investigate whether muscle-specific RNAi for proteasome components induces adaptive stress responses in distant tissues, fractions from thoraces (composed of skeletal muscle) and heads (composed mostly of retinas and brains) from Mhc>Prosβ1^{RNAi} and control Mhc>white^{RNAi} flies were analyzed. Muscle-specific RNAi for Prosβ1 led to limited changes in the age-related accumulation of poly-ubiquitinated proteins in detergent-insoluble fractions of muscle, consistent with the induction of compensatory local (cell-autonomous) adaptive responses to Prosβ1^{RNAi}. However, muscle-specific Prosβ1^{RNAi} cell-non-autonomously increased the degradation of proteasome substrates during aging in non-muscle tissues, as assessed from the analysis of poly-ubiquitinated proteins in detergent-insoluble fractions from heads at 10, 30, and 60 days of age.

To further probe this cell-non-autonomous stress response, the effect of muscle-specific RNAi for Ter94 was examined. Different from Prosβ1^{RNAi}, Ter94^{RNAi} worsened proteostasis in muscle, suggesting that local adaptive responses do not completely compensate for the decline in protein quality due to Ter94 RNAi. However, as observed for Prosβ1^{RNAi}, muscle-specific Ter94^{RNAi} improved protein quality control cell non-autonomously, as indicated by the lower age-related accumulation of poly-ubiquitinated proteins in detergent-insoluble fractions from heads, compared to control RNAi (white^{RNAi} and vermillion^{RNAi}) and to transgene-alone controls.

Similar results were obtained via muscle-specific Prosβ1^{RNAi} expression obtained with the drug-inducible Act88F-GeneSwitch-Gal4 driver, which is specific for thoracic indirect flight muscles (Robles-Murguia, et al. (2019) npj Aging Mech Dis 5:6), i.e., muscle-specific expression of Prosβ1^{RNAi} decreased the levels of poly-ubiquitinated proteins in Drosophila heads compared to uninduced controls and to white^{RNAi}.

To test whether this response is induced only by Prosβ1^{RNAi} and Ter94^{RNAi} or is generally induced by RNAi for components of the 20S proteasome, the effect of muscle-specific RNAi was examined for other proteasomal components. As observed for Prosβ1^{RNAi}, RNAi for Prosa3, 7 Prosa4, Prosa5, Prosa6, Prosβ2, Prosβ4, and Prosβ5 led to a reduction in the age-related accumulation of poly-ubiquitinated proteins in detergent-insoluble fractions from heads, indicating that this cell-non-autonomous improvement in protein quality can be induced by targeting several 20S proteasomal components in muscle. Interestingly, autophagy does not seem to be responsible for these effects given that Atg8 conversion and levels were not substantially regulated. Altogether, these findings indicate that local perturbation of proteasome function in thoracic skeletal muscle induces a systemic adaptive response that improves the degradation of proteasome substrates in non-muscle tissues.

To corroborate these findings, immunostaining was used to test whether drug-induced, muscle-restricted induction of Prosβ1^{RNAi} regulates protein quality control in the retina, which is characterized by the accumulation of poly-ubiquitinated protein aggregates during aging (Demontis & Perrimon (2010) Cell 143(5):813-825). Interestingly, muscle-specific Prosβ1^{RNAi} led to a decrease in the age-related accumulation of protein aggregates in the retina, further confirming that proteasome stress in muscle improves the degradation of proteasome substrates in distant, non-muscle tissues during aging.

### Example 3: Amyrel is a Myokine that is Induced by Proteasome Stress and Regulates Systemic Protein Quality Control

The transcriptional response induced by proteasome stress in muscle included secreted proteins, which indicated that muscle-secreted signaling factors (myokines) are responsible for the cell non-autonomous regulation of protein quality control. In this respect, it was determined whether myokine gene expression changed in response to proteasome stress by comparing RNA sequencing data from muscle with Prosβ1^{RNAi}, Prosβ5^{RNAi} and Ter94^{RNAi} to control RNAi. Several myokines were regulated in an RNAi-specific manner whereas others were consistently induced by Prosβ1^{RNAi}, Prosβ5^{RNAi} and Ter94^{RNAi} but not by GFP^{RNAi}, compared to white^{RNAi}. On this basis, it was determined whether any of the myokines consistently induced by proteasome stress were responsible for systemic stress signaling. Specifically, it was examined whether RNAi and/or overexpression of stress-regulated myokines modulates the degradation of proteasome substrates in heads during aging.

To this purpose, detergent-soluble and detergent-insoluble fractions were analyzed, which were obtained at 10, 30, and 60 days from heads of flies with muscle-specific overexpression and RNAi for some of the myokines that were most significantly regulated by proteasome stress: CG8093, Amyrel, and Ag5r2 (upregulated), and CG4666, takeout (to), and Akh (downregulated).

Overall, Amyrel (a secreted amylase enzyme; Claisse, et al. (2016) Insect Biochem. Mol. Biol. 75:70-77) consistently regulated protein quality control in a manner coincident with its transcriptional upregulation by proteasome stress. Specifically, Amyrel overexpression in muscle reduced the age-related accumulation of proteasome substrates in head tissues (consisting primarily of retinas and the brain) whereas muscle-specific Amyrel RNAi increased it. Interestingly, preservation of protein quality control by Amyrel does not seem to depend on autophagy, given that Atg8 conversion and levels were not significantly modulated. Conversely, other myokines had only minor effects or led to outcomes inconsistent with their modulation by proteasome stress. Interestingly, although muscle-specific Amyrel overexpression promoted protein quality in head tissues, it did not regulate proteostasis in skeletal muscle, indicating that skeletal muscle does not respond to Amyrel-mediated signaling. Consistent with its roles in the systemic regulation of protein quality control, GFP-tagged Amyrel expressed by skeletal muscle was also detected in the fly circulation (hemolymph).

To test whether Amyrel is indeed a key myokine acting downstream of Prosβ1^{RNAi} it was determined whether muscle-specific Amyrel^{RNAi} impedes the systemic response induced by Prosβ1^{RNAi}. In agreement with this hypothesis, the levels of poly-ubiquitinated proteins were higher in the detergent-insoluble fractions of heads from flies with muscle-specific Prosβ1^{RNAi}+Amyrel^{RNAi} compared to Prosβ1^{RNAi}+control^{RNAi}. Together, these findings indicate that Amyrel is a key myokine that contributes to the Prosβ1^{RNAi}-induced, cell-non-autonomous preservation of protein quality control during aging.

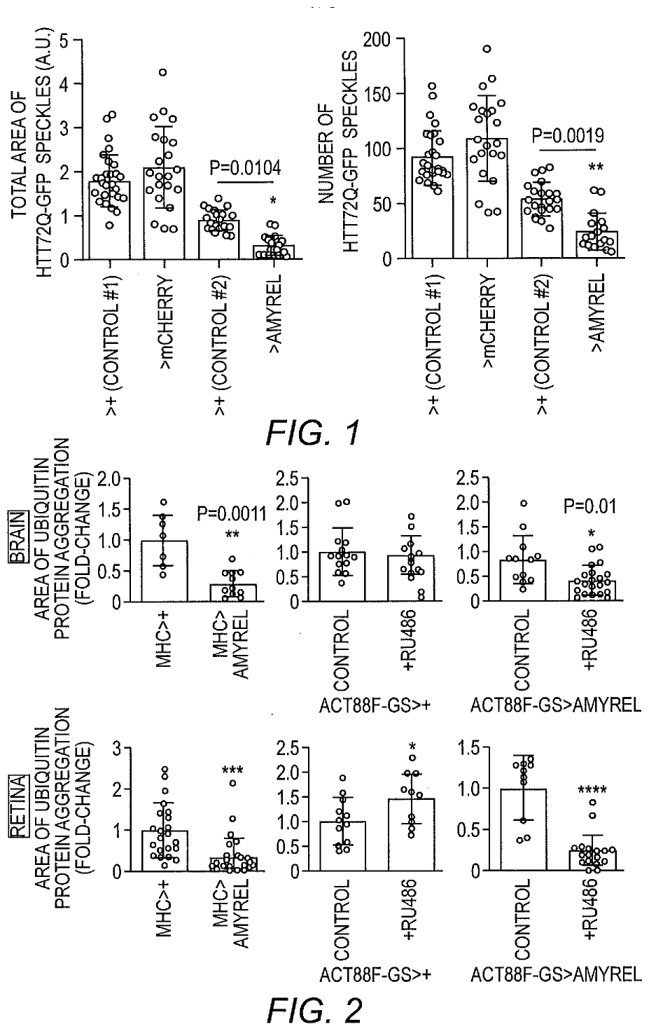

Age-related loss of protein quality control is accentuated by the concomitant expression of disease-associated aggregation-prone proteins (Balch, et al. (2008) Science 319:916-919; Douglas & Dillin (2010) J. Cell Biol. 190(5):719-29). On this basis, it was determined whether muscle-specific Amyrel overexpression regulates proteostasis in heads of flies with pathogenic Huntingtin (Htt-polyQ120) expression in the retina. Interestingly, muscle-derived Amyrel reduced the levels of polyubiquitinated proteins also in this context, indicating that Amyrel improves protein quality control also in neurodegeneration-like conditions. To further probe the capacity of Amyrel to maintain protein quality control, it was determined whether Amyrel affects pathogenic GFP-tagged Huntingtin-polyQ72. Such aggregates increase in an age-dependent manner in the Drosophila retina but less so in response to Amyrel overexpression when compared to controls (FIG. 1).

The effect of Amyrel on the age-related neurodegeneration induced by mutant tau^{V337M} was also analyzed. The tau^{V337M} mutant is known to cause frontotemporal dementia in humans (Nacharaju, et al. (1999) FEBS Lett. 447(2-3):195-9). In Drosophila, tau^{V337M} induces retinal degeneration during aging, as exemplified by the appearance of a rough eye phenotype due to the loss and derangement of photoreceptor neurons. However, Amyrel overexpression largely prevented such age-related neurodegeneration. Together, these studies indicate that muscle-derived Amyrel is a key stress-induced myokine that promotes the degradation of proteasome substrates during aging and prevents neurodegeneration induced by pathogenic proteins.

### Example 4: Proteasome Stress Induces Amyrel Expression via C/EBP Transcription Factors

The transcriptional mechanisms responsible for increased Amyrel expression in muscle in response to proteasome stress were subsequently examined. In particular, the proximal 1 kb Amyrel promoter region was scanned to identify transcription factor binding sites. These included two binding sites for C/EBP (CCAAT/enhancer-binding protein) transcription factors, which are known to be modulated by cellular stress in other contexts (Hattori, et al. (2003) Oncogene 22:1273-1280; Hungness, et al. (2002) J. Cell. Physiol. 192(1):64-70; Shim & Smart (2003) J. Biol. Chem. 278(22):19674-81).

To test whether C/EBPs sense proteasome stress in skeletal muscle, the transcriptional changes induced by RNAi for CG6272/Irbp18 was examined. Notably, CG6272/Irbp18 is the Drosophila homolog of C/EBPγ. Due to the lack of a transactivation domain, C/EBPγ acts as a dominant negative inhibitor via dimerization with other C/EBPs (C/EBPβ and C/EBPδ). Therefore, RNAi for CG6272 (C/EBPγ) promotes the transcriptional activity of C/EBPβ/δ (Lekstrom-Himes & Xanthopoulos (1998) J. Biol. Chem. 273:28545-48).

Consistent with a role of C/EBPs in mediating the transcriptional response to proteasome inhibition, there was substantial overlap (R²=0.447 for all genes, and R²=0.771 for p<0.05) in the gene expression changes induced by CG6272^{RNAi} versus those induced by Prosβ1^{RNAi} and Prosβ5^{RNAi}, but not by GFP^{RNAi}. Moreover, functionally related groups of genes are induced by CG6272^{RNAi} versus those induced by Prosβ1^{RNAi} and Prosβ5^{RNAi}, including proteases/peptidases and secreted factors. Further analysis of the 37 muscle-secreted factors (myokines) regulated by proteasome stress further indicated that 28 of these (75%) were consistently and significantly regulated also by CG6272^{RNAi}, including Amyrel. Similar results were also obtained with drug-induced expression of CG6272^{RNAi} by using Act88F-GS-Gal4. To further corroborate these findings, the function of Slbo, the Drosophila homolog of mammalian C/EBPβ/δ, was tested. As expected, based on the analysis of CG6272 (C/EBPγ), slbo overexpression (slbo^{OE}) led to an increase in Amyrel gene expression. Altogether, these findings indicate a key role for C/EBPs in mediating the transcriptional response to proteasome stress in skeletal muscle, which in turn leads to both local and systemic adaptive responses.

### Example 5: Muscle-Derived Amyrel Promotes the Degradation of Proteasome Substrates in the Retina and Brain During Aging

It was observed that muscle-derived Amyrel is a key myokine induced by proteasome stress that promotes the degradation of proteasome substrates in distant tissues. Specifically, muscle-specific Amyrel overexpression reduced the age-related increase in polyubiquitinated proteins in detergent-insoluble fractions of head tissues, which are composed primarily of brains and retinas. On this basis, immunostaining was used to further probe these findings.

As previously observed (Demontis & Perrimon (2010) Cell 143(5):813-825), poly-ubiquitin protein aggregates accumulate in the retina and brain during aging in Drosophila. However, muscle-specific overexpression of Amyrel with either Mhc-Gal4 or Act88F-GS-Gal4 significantly reduced the amount of poly-ubiquitin protein aggregates detected in old age in the retina and brain compared to isogenic controls (FIG. 2). These results are in agreement with the biochemical analyses.

To further test the relevance of Amyrel downstream of Prosβ1^{RNAi}, the levels of poly-ubiquitinated proteins in the brains and retinas of flies with muscle-specific Prosβ1^{RNAi}+Amyrel^{RNAi} compared to Prosβ1^{RNAi}+control_{RNAi} were examined. Consistent with the biochemical analyses, it was found that Amyrel is necessary for the improvement of protein quality control in brains and retinas in response to muscle-specific Prosβ1^{RNAi}. Altogether, these findings indicate that muscle-produced Amyrel preserves protein quality control in the brain and retina during aging in response to proteasome stress in muscle.

### Example 6: Muscle-Derived Amyrel Induces Chaperone and Protease Gene Expression in Head Tissues and Delays Age-related Functional Decline

To dissect the mechanisms by which Amyrel regulates proteostasis, the head transcriptome of flies with muscle-specific Amyrel overexpression was examined and compared to controls. There were several genes involved in proteostasis that were induced by Amyrel compared to isogenic controls, including several chaperones and proteases. In addition, there were other gene categories relevant for aging and neurodegeneration, such as glutathione and lipid metabolism, that were enhanced in response to muscle-specific Amyrel overexpression.

Proteases are known to compensate for proteasome dysfunction and may therefore contribute to protein quality control in response to Amyrel. Consistent with this, pathogenic Huntingtin aggregation was increased by RNAi for the Amyrel-induced proteases CG9733 and CG7142.

Chaperones exert anti-aging functions by refolding misfolded proteins, by shielding aggregation-prone proteins from interacting with endogenous proteins, and by routing them to proteasomal degradation. On this basis, Amyrel-induced heat shock proteins may promote protein quality control by chaperoning and degrading proteasomal substrates, such as poly-ubiquitinated proteins and aggregation-prone proteins. On this basis, the role of the small heat shock protein Hsp23 was examined. Notably, the Hsp23 gene was the highly induced (>8-fold) gene by Amyrel. Consistent with a role of Hsp23 in regulating protein quality control downstream of Amyrel, transgenic Hsp23 overexpression reduced pathogenic Huntingtin aggregation, as observed in response to Amyrel. Conversely, RNAi for Hsp23 and other chaperones induced by Amyrel (Hsp68) led to an increase in the area of Huntingtin-polyQ72 aggregates.

It was subsequently determined whether Hsp23 overexpression in the brain protects from the age-related accumulation of poly-ubiquitinated proteins in detergent-insoluble fractions. Consistent with this model, drug-induced expression of Hsp23 with elav-GS-Gal4 largely prevented the age-related increases in ubiquitinated proteins in head tissues compared to controls. These findings indicate that the small heat shock protein Hsp23 is a key Amyrel-induced gene that promotes proteostasis.

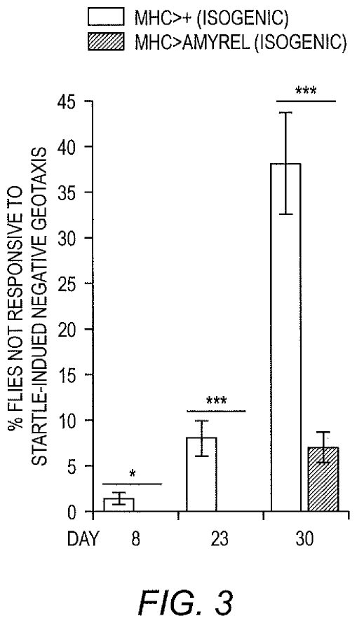

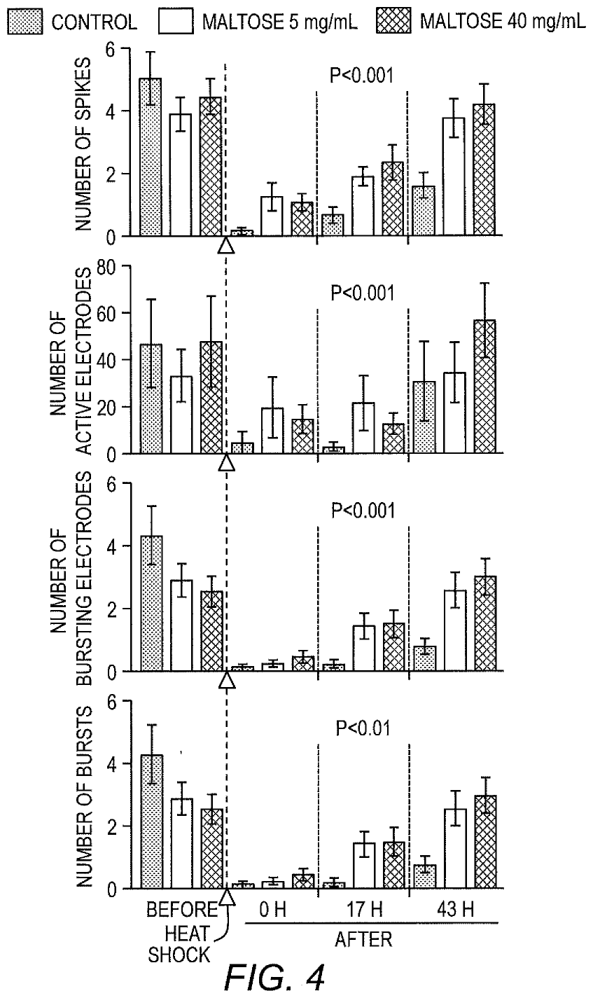

In the brain and retina, preservation of protein quality control is necessary for ensuring neuronal activity and survival (Douglas & Dillin (2010) J. Cell Biol. 190(5):719-729). Previously, neuron-restricted expression of small heat shock proteins was found to preserve startle-induced locomotion with aging, as assessed with negative geotaxis assays (Morrow, et al. (2004) FASEB J. 18(3):598-9). On this basis, because Amyrel induces the expression of small heat shock proteins, it was assessed whether Amyrel preserves startle-induced locomotion with aging. Interestingly, the percentage of flies unable to respond to stimulation increases with aging but less so with muscle-specific Amyrel overexpression compared to isogenic controls (FIG. 3). Because Amyrel does not improve proteostasis in skeletal muscle, preservation of startle-induced negative geotaxis likely reflects the action of Amyrel on the nervous system. Together, these findings indicate that Amyrel preserves neuronal function during aging by inducing the expression of protective genes, including proteases and chaperones such as Hsp23.

### Example 7: Muscle Amyrel Increases Maltose Levels

Having established that Amyrel expression increases in response to proteasome stress in muscle and that muscle-derived Amyrel promotes the degradation of proteasome substrates in the brain and retina via the transcriptional induction of protective target genes, Amyrel's mechanism of action was further examined.

Because muscle-produced Amyrel is detected both within skeletal muscle and in the circulation, it was posited that Amyrel's amylase activity in one or more of these compartments may produce a systemic factor that promotes proteostasis. In agreement with the importance of Amyrel's amylase activity, overexpression of other amylases (Amy-d and Amy-p) also reduced the levels of fluorescent Huntingtin-polyQ72-GFP aggregates in the retina similar to what is observed with Amyrel. These findings indicate an important role for the enzymatic activity of Amyrel in the modulation of protein quality control during aging.

As observed for amylases in the digestive system, stress-induced muscle Amyrel may produce maltose and other disaccharides via the enzymatic degradation of polysaccharides and oligosaccharides. To test whether this occurs, the effect of muscle-specific Prosβ1^{RNAi} was examined. This analysis indicated that whole body maltose levels were higher, compared to control white^{RNAi}, whereas glucose levels were not consistently regulated. In addition, there was an increase in whole body and circulating (hemolymph) maltose upon Amyrel overexpression, in comparison with isogenic controls, in parallel with an increase in amylase activity. Altogether, these findings indicate that Amyrel increases maltose levels, as expected based on its enzymatic activity.

### Example 8: Maltose and Maltose Transporters Promote Protein Quality Control

Because Amyrel increases maltose levels, it was determined whether maltose is a key modulator of protein quality control produced in response to proteasome stress. For these experiments, Drosophila S2R+ cells were treated with recombinant porcine amylase or maltose. The effect of such treatments was assessed by western blot analysis after heat shock, which induces protein misfolding and increases the degradation burden for the proteasome (Douglas & Dillin (2010) J. Cell Biol. 190(5):719-729; Balch, et al. (2008) Science 319:916-919). Similar to results obtained with transgenic Amyrel overexpression in vivo in Drosophila, treatment with recombinant amylase reduced the accumulation of poly-ubiquitinated proteins in detergent-insoluble fractions upon heat shock. Interestingly, similar results were obtained by treating S2R+ cells with maltose, indicating that maltose is an important modulator of protein quality.

Because maltose improves protein quality control, it was determined whether maltose transmembrane transporters induce similar responses. In this respect, a Huntington disease model was used to test the role of maltose transporters in protein quality control in Drosophila. Specifically, it was examined whether RNAi for Slc45 regulates the levels of Huntingtin-polyQ72-GFP aggregates in the Drosophila retina, as was observed for Amyrel. For these studies, RNAi lines for the maltose transporters Slc45-1 and Slc45-2 (Meyer, et al. (2011) J. Cell Sci. 124(Pt 12):1984-91; Vitayska & Wieczorek (2013) Mol. Aspects Med. 34(2-3):655-660) were used. In addition, the unrelated trehalose transporter Tret1-1 was examined. Expression of these transporters was driven in the retina with GMR-Gal4. Compared to control interventions, RNAi for Slc45-1 and Slc45-2 significantly increased Huntingtin-polyQ72-GFP aggregates, opposite to what is found with Amyrel overexpression and similar to RNAi for the puromycin-sensitive aminopeptidase (PSA), which is necessary for the degradation of pathogenic Huntingtin (Menzies, et al. (2010) Hum. Mol. Genet. 19(23):4573-4586). Moreover, RNAi for Slc45-1 worsened age-related neurodegeneration induced by tau^{V3337M} compared to control mCherry RNAi. Overall, these findings indicate a key role of maltose transporters in protein quality control.

### Example 9: RNAi for Maltose Transporters Reduces the Expression of Amyrel-Target Genes in the Brain

Maltose and disaccharides have been reported to act as chemical chaperones that stabilize membrane structure and promote protein folding (Kaplan & Guy (2004) Plant Physiol. 135:1674-84; Mensink, et al. (2017) Eur. J. Pharm. Biopharm. 114:288-295; Levy-Sakin, et al. (2014) PLoS ONE 9(2):e88541). Therefore, intracellular transport of maltose may affect protein quality control via the chemical chaperone properties of maltose. In addition, maltose intracellular transport by Slc45 may mediate Amyrel-induced gene expression changes. Moreover, because Slc45-2 is highly expressed in the brain but has little or no expression in skeletal muscle, its expression pattern may explain why protein quality control improves in the brain but not in muscle in response to Amyrel. To test this, qRT-PCR analysis was conducted to determine whether RNAi for Slc45-2 reduces the expression of Amyrel-induced genes in the brain. In parallel with a decline in Slc45-2 mRNA levels, there was a decrease in the expression of key Amyrel-induced chaperones (Hsp23, Hsp26, Hsp27), indicating that Slc45-2 is necessary for Amyrel-induced target gene expression in the brain.

On this basis, it was assessed whether Slc45-2 RNAi regulates the age-related accumulation of poly-ubiquitinated proteins in detergent-insoluble fractions in head tissues during aging. S1c45-2 RNAi induction with elav-Gal4 led to an increase in poly-ubiquitinated proteins with aging, compared to control white^{RNAi} and mCherry^{RNAi}, as expected based on the decreased expression of chaperones by Slc45-2RNAi. Similar results were obtained with drug-induced expression of Slc45-2^{RNAi} with elav-GS-Gal4, compared to controls.

Together, these studies indicate that maltose transporters are necessary for protein quality control in Drosophila as it was expected based on the role of maltose and of the maltose-producing enzyme Amyrel in this process. Accordingly, these studies identify an unanticipated pathway for protein quality assurance that originates from maltose production by the muscle amylase enzyme Amyrel and requires Slc45 maltose transporters for chaperone gene expression and protein quality control in the brain during aging.

### Example 10: Maltose Preserves Protein Quality in Human Cells in a SCL45-Dependent Manner

To further probe this model, the effect of maltose was examined in human HEK293 cells, which have a neuronal origin (Lin, et al. (2014) Nat. Commun. 5:4767; Shaw, et al. (2002) FASEB J. 16(8):869-71). As observed for S2R+ cells, maltose treatment significantly prevented the heat shock-induced accumulation of polyubiquitinated proteins in detergent-insoluble fractions, compared to control treatments. These controls included iso-osmolar (NaCl) and iso-energetic (glucose) treatments, which did not fully recapitulate the preservation of protein quality control seen with maltose. Together, these findings indicate that the disaccharide maltose improves protein quality control downstream of Amyrel via mechanisms different from osmotic stress and metabolic utilization.

On the basis of these results, it was subsequently tested whether maltose transporters affect protein quality control also in human HEK293 cells as observed in Drosophila. Accordingly, HEK293 cells were treated with either siRNAs for the maltose transporters SLC45A3 and SLC45A4 (the sole SLC45 family members expressed in HEK293 cells), or with control NT siRNAs. Upon heat shock, there was an accumulation of poly-ubiquitinated proteins in the detergent-insoluble fractions, which was largely prevented by maltose in HEK293 cells treated with control NT siRNAs, but less so in cells treated with SLC45A3/4 siRNAs. Altogether, these studies indicate that maltose preserves protein quality in human cells in a SCL45-dependent manner.

### Example 11: Maltose Preserves Protein Quality and Neuronal Activity in Human Cortical Brain Organoids Challenged by Heat Shock

Organoids are emerging as important disease models for aging and brain research (Hu, et al. (2018) Cell 175(6):1591-1606; Qian, et al. (2016) Cell 165(5):1238-1254). Thus, it was determined whether maltose regulates protein quality control in heat-shocked organoids as observed in human cells. There was no modulation of poly-ubiquitinated proteins in detergent-soluble and insoluble fractions in maltose-treated versus control cells in non heat-shocked conditions. However, heat shock significantly increased the levels of poly-ubiquitinated proteins in detergent-insoluble fractions and, as observed for HEK293 cells, maltose could partly prevent this increase. Similar results were obtained via immunostaining. Specifically, while there was no change in the levels of p62 and poly-ubiquitinated proteins in control conditions, maltose significantly decreased p62 and ubiquitin immunoreactivity in heat-shocked organoids, compared to controls.

Gene expression changes induced by maltose was also examined in human cortical brain organoids. RNA-seq indicated that maltose prevented some of the gene expression changes induced by heat shock. Interestingly, treatment with maltose promoted the expression of several gene clusters involved in protein quality control, such as components of the proteasome and proteases/peptidases. Moreover, mRNA and proteins levels of CRYA (a-crystallin, homologous to Drosophila Hsp23) were increased by maltose treatment in human cortical organoids, consistent with findings in Drosophila brains and retinas.

Because preservation of protein quality control typically corresponds to improvements in brain functions, it was determined whether the neural activity of organoids was improved by treatments with maltose. Measurements with microelectrode arrays (MEAS) indicated that the number of active electrodes, bursting electrodes, and network bursts was not modulated by maltose at steady state but was significantly preserved by maltose immediately after and at 17 hours and 43 hours after heat shock (FIG. 4). Interestingly, levels of maltose as low as 5 mg/mL were effective in preserving neuronal activity (FIG. 4), coincident with the preservation of protein quality. Altogether, these findings demonstrate that maltose has an evolutionary conserved role in preserving protein quality control in the brain.

What is claimed is:

1. A method for preventing or treating a neurodegenerative disease or condition comprising administering to a subject in need thereof an effective amount of an amylase or maltose thereby preventing or treating neurodegenerative disease or condition.

2. The method of claim 1, wherein the amylase is administered in the form of an isolated amylase protein.

3. The method of claim 1, wherein the amylase is administered in the form of an amylase encoding nucleic acid molecule.

4. The method of claim 3, wherein the amylase encoding nucleic acid molecule is inserted in a viral vector.

5. The method of claim 1, wherein the neurodegenerative disease or condition is a neurodegenerative proteinopathy.

6. The method of claim 5, wherein the neurodegenerative proteinopathy is Huntington's disease, Alzheimer's disease or Parkinson's disease.

7. The method of claim 1, wherein the effective amount of amylase or maltose reduces aggregation-associated or misfolded protein-associated proteotoxicity, induces transcription of chaperones and proteases, promotes degradation of proteasome substrates, or preserves protein quality under stress conditions in a subject.

8. The method of claim 1, wherein the maltose is administered in the form of a pharmaceutical composition consisting of an effective amount of maltose in admixture with a suitable carrier.
